(19) United States
(12) Patent Application Publication
(10) Pub. No.: US 2012/0109168 A1
(43) Pub. Date: May 3, 2012

(54) BLOOD VESSEL ATTACHMENT FOR A VASCULAR PROSTHESIS

(75) Inventor: Dietmar Gerhardt, Kropp (DE)

(73) Assignee: S & C SYSTEMS GMBH, Luebeck (DE)

(21) Appl. No.: 13/060,356

(22) PCT Filed: Aug. 21, 2009
(86) PCT No.: PCT/DE2009/001174

§ 371 Date: Jan. 17, 2012

(30) Foreign Application Priority Data
Aug. 23, 2008 (DE) 10 2008 039 523.4

(51) Int. Cl. A61B 17/11

(52) U.S. Cl. 606/153

(57) ABSTRACT

A blood vessel connection for a vessel prosthesis includes a connection part, which may be introduced in sections into an open end of a blood vessel and forms a flow channel from the blood vessel to a vessel prosthesis connected on the connection part. The blood vessel connection has a clamping component wherein the blood vessel may be fastened on the connection part.

### CROSS-REFERENCE TO RELATED APPLICATIONS

This application is a Section 371 of International Application No. PCT/DE2009/001174, filed Aug. 21, 2009, which was published in the German language on Mar. 4, 2010, under International Publication No. WO 2010/022705 A1 and the disclosure of which is incorporated herein by reference.

### BACKGROUND OF THE INVENTION

It is common to replace sections of blood vessels which have been damaged due to injury or illness, in particular arteries, e.g. given an aneurysm, by vessel prostheses. These vessel prostheses are typically designed in a flexible-tube-like manner from biocompatible plastics, for example polytetrafluoroethylene, wherein their cross-sectional dimensions correspond essentially to the cross-sectional dimensions of the blood vessel sections to be replaced.

The connection of the vessel prosthesis to the open ends of the blood vessel has been effected by suturing until now. This is relatively time-consuming and is therefore of a burden to the operator as well as to the patient. Moreover, there is the danger that a bleeding occurs in the sutured region as a result of leakage of the vessel wall which is perforated by the incision channels, or of an embolus arising there at a cross-sectional transition.

### BRIEF SUMMARY OF THE INVENTION

Against this background, it is the object of the invention to provide a connection of a blood vessel to a vessel prosthesis, with which the danger of one of the complications mentioned above occurring is reduced in comparison to a sutured connection. Moreover, a vessel prosthesis with this connection should be able to be fastened on a blood vessel in a significantly quicker manner.

This object is achieved by a blood vessel connection for a vessel prosthesis comprising a connection part which in sections, with a tubular section, may be introduced into an open end of a blood vessel and forms a flow channel from the blood vessel to a vessel prosthesis connected on the connection part, a sleeve-like clamping component for arrangement on the outer side of the tubular section, with which the blood vessel may be fastened on the connection part and with a fastening sleeve for fixing the vessel prosthesis, characterised in that a cavity of the connection part, which forms the flow channel through the connection part, is widened in a shoulder-like manner in a section which connects to the tubular section, wherein the fastening sleeve may be screwed into the widened section and with the widened section is dimensioned such that the inner wall of the vessel prosthesis is flush with the inner wall of the flow channel formed by the connection part, and wherein the tubular section and the clamping component comprise a multitude of openings in each case distributed about the periphery.

Advantageous further embodiments of this blood vessel connection are to be deduced from the subsequent description, claims and drawings.

For the connection of a blood vessel to a vessel prosthesis, the invention envisages a blood vessel connection having a connection part. The connection part in sections may be introduced into the open end of the blood vessel, which arises after the removal of a damaged vessel section, and forms a flow channel from the blood vessel to the vessel prosthesis, which is likewise connected to the connection part. The blood vessel connection comprises a clamping component for fastening the blood vessel onto the connection part. With this clamping component a force, which fixes the blood vessel onto the connection part with a force-fit and/or form-fit, is exerted onto the outer side of the blood vessel and usefully over its complete periphery. Optionally, a fixed connection of the blood vessel to the vessel prosthesis, which is created in this manner, may be improved to the extent that the blood vessel is additionally bonded to the connection part and/or to the clamping component with a biocompatible adhesive.

Advantageously thus, with the blood vessel connection according to embodiments of the invention, a connection of a vessel prosthesis to a blood vessel is possible, without having to suture the implant to the vessel. The duration of the operation on applying the vessel prosthesis is thus significantly reduced. This is particularly advantageous if the vessel prosthesis is to be inserted into those blood vessels which supply vital organs with blood. The blood supply to these organs is interrupted for a significantly shorter time with the use of the blood vessel connection according to embodiments of the invention.

A further advantage of the blood vessel connection according to embodiments of the invention is to be seen in the fact that the healthy part of the blood vessel is not damaged, which is in contrast to the suturing of the blood vessel to the vessel prosthesis, which has been common until now. Accordingly, with the application of the blood vessel connection according to embodiments of the invention, the danger of an aneurysm forming in vessel sections connecting to the vessel prosthesis is considerably smaller.

Usefully, the section of the connection part which is to be introduced into the blood vessel is designed in a tubular manner, wherein this tubular section of the connection part typically has an outer cross section which corresponds essentially to the inner cross section of the blood vessel. For fastening the blood vessel on the section of the connection part which is introduced therein, the blood vessel connection preferably comprises a sleeve-like clamping component, which is provided for arrangement on the outer side of the tubular section of the connection part. That is, similarly to the use of a hose clip, the blood vessel with this design is pressed over its complete periphery by the clamping component onto the section of the connection part which is located in the blood vessel. This ensures that no undesired blood loss may occur in the region of the connection of the blood vessel to the blood vessel connection.

A particularly good sealing of this connection may be achieved if the sleeve-like clamping component envelops the blood vessel in the section, in which it overlaps the introduced connection part, essentially over the whole length of this section. Accordingly, the clamping component fixedly clamps the blood vessel onto the connection part, preferably in a region which extends in the longitudinal direction of the blood vessel or of the connection part, essentially from the distal end of the connection part, which is to say the end of the connection part which is introduced into the blood vessel, up to the open end of the blood vessel.

This is advantageous inasmuch as the blood vessel is held on the connection part with a sufficiently large clamping force, without having to fear a damage to the blood vessel wall due to this clamping force, since the surface pressing on the blood vessel is comparatively small on account of the thus created large overlapping region or bearing contact region of the clamping component on the blood vessel. A further advantage is to be seen in the fact that the clamping component, due to the fact that it comes to bear on the blood vessel in the complete region in which the blood vessel overlaps the connection part, ensures that no dead spaces may form between the blood vessel and the connection part, in which dead spaces blood clots could otherwise settle.

Preferably, the clamping component is designed of two parts. Usefully, the division of the clamping component is hereby effected in a plane parallel to the longitudinal axis of the clamping component, and particularly advantageously in a common plane with the longitudinal axis of the clamping component, so that the clamping component is divided approximately into two halves. Accordingly, the clamping component with this design consists of two essentially shell-like parts, which are arranged in a simple manner on the outer side of the blood vessel, and may there be joined together while forming a fixed connection.

In this context, one preferably envisages the parts of the clamping component being designed for forming a locking connection. The two parts of the clamping component may thus preferably comprise locking parts in the region of their joining edges to the respective other part of the clamping component, these locking parts being able to be brought into engagement with one another in a manner such that a force-fit and/or form-fit connection of the two parts of the clamping components arises after the locking-in of these locking parts.

For fastening the two parts of the clamping component, advantageously at least one groove extending around the outer periphery of the clamping component may be formed on the clamping component. That is, the two parts of the clamping component may in each case comprise a groove on its outer side, and this groove in the assembled condition of the two parts results in a groove running around the complete periphery of the clamping component. This groove may serve for receiving a fastening thread which, after the two ends of the fastening thread have been tied to one another, holds the two parts of the clamping component together in a fixed manner. A particularly secure connection of the two parts of the clamping component is possible when these parts are connected to one another with locking parts formed on these, as well as with one, preferably several fastening threads which are arranged in the manner described above. Correspondingly more grooves are provided on the outer periphery of the clamping component for more fastening threads.

A further preferred design of the blood vessel connection according to the invention envisages the two parts of the clamping component being connected by a hinge. That is, the two parts of the clamping component may be connected at two oppositely lying joining edges in an articulated and preferably non-releasable manner by the hinge, wherein the two parts at their respective other joining edge are releasably connectable to one another, preferably with locking parts. The advantage with this design is the fact that the clamping component as a single component may be arranged around the blood vessel and fastened there in a significantly simpler manner than with a two-part design with two separate parts.

In order to prevent a slippage of the clamping component in the axial direction relative to the connection part, the clamping component and the connection part are advantageously connectable to one another with a form fit. Thus, the clamping component preferably comprises at least one radially inwardly directed projection, which is envisaged for engagement into at least one recess formed on the outer side of the connection part. Alternatively or additionally, at least one radially outwardly extending projection, which engages into at least one recess formed on the inner side of the clamping component, may be provided on the connection part. In order not to injure the blood vessel on forming such a form fit, the clamping component is usefully dimensioned in a manner such that it projects beyond the open end of the blood vessel in the axial direction, wherein the form fit between the clamping component and the connection part is created in this region projecting beyond the blood vessel. That is, the clamping component comprises a clamping section for clamping the blood vessel, and a connection section connecting in the axial direction for engagement with the connection part.

A slippage of the blood vessel in the axial direction relative to the connection part and the clamping component is preferably prevented by the fact that at least one radially outwardly directed projection, which extends preferably around the complete periphery of the connection part, is formed on the outer side of the section of the connection part, which may be introduced into the blood vessel. When the blood vessel is clamped between the connection part and the clamping component, this projection amid the displacement of blood vessel tissue presses into the wall of the blood vessel transversely to a possible axial movement direction of the blood vessel. Usefully, the projection is dimensioned and designed such that the wall of the blood vessel may not be perforated with it. With a reduced pressing pressure, the retention of the blood vessel by the clamping component may be improved by one or more such projections which form a transverse grooving.

The connection part is preferably designed in a manner such that its outer cross section in the region of the tubular section tapers in the direction of its free end. Hereby, one usually envisages the connection part, at least in the region in which it is overlapped by the blood vessel, having a constant inner cross section which corresponds essentially to the inner cross section of the blood vessel, and only the wall thickness of the connection part reduces preferably in a continuous manner in the direction of the distal end of the connection part. This is advantageous inasmuch as thereby at most a very small and preferably almost no cross-sectional change of the flow path in the blood vessel arises with the transition from the connection part to the blood vessel connected thereto, so that one may rule out as much as possible thromboses arising at this location. A further advantage of this design is to be seen in the fact that it simplifies the introduction of the connection part into the blood vessel.

A further preferred design of the blood vessel connection according to an embodiment of the invention envisages a cavity of the connection part, which forms the flow channel through the connection part, widening in a shoulder-like manner in a section which connects to the tubular section which may be introduced into the blood vessel. Hereby, the blood vessel connection comprises a fastening sleeve for fixing the vessel prosthesis, which may be screwed into the widened section of the connection part. For this, an inner thread is provided on the inner periphery of the widened section, and a corresponding outer thread is formed on the outer periphery of the fastening sleeve. Thus the fastening sleeve, and with this the vessel prosthesis, may be fastened in a simple manner on the connection part. Advantageously, this fastening may be effected already before the operation for applying the vessel prosthesis in a blood vessel, which further shortens the duration of this operation.

Usefully, the widened section of the connection part and the fastening sleeve, which may be screwed therein, are dimensioned in the radial direction such that the inner wall of the vessel prosthesis, which is connected to the connection part by the fastening sleeve and whose inner cross section typically essentially corresponds to the inner cross section of the blood vessel, is flush with the inner wall of the flow channel formed by the connection part, so that the blood vessel, the connection part and the vessel prosthesis form an essentially shoulderless, smooth flow channel with a constant inner cross section, in order to prevent an embolus formation within the blood vessel connection according to the invention.

The vessel prosthesis may be fastened directly on the fastening sleeve. Apart from this, it is also possible to clamp the vessel prosthesis with the fastening sleeve in the connection part. In this context, a design is preferred with which the fastening sleeve is dimensioned in the axial direction in a manner such that a vessel prosthesis, which is led through the fastening sleeve and which overlaps the end of the fastening sleeve which engages into the connection part in the screwed-in position, may be clamped between this end-face of the fastening sleeve and an end-side shoulder of the connection part. With this design, usefully the wall of the fastening sleeve, in the region of its end engaging into the connection part, is spaced from the wall of the connection part which is arranged around this on the outer side, so that an intermediate space is formed in this region between the walls of the connection part and the fastening sleeve, and this intermediate space may serve for receiving the section of the vessel prosthesis which overlaps the fastening sleeve. That is, the fastening sleeve in an axial region has a smaller outer diameter than the inner diameter of the widened section. The fastening sleeve in an axially connecting region has an outer diameter corresponding to the inner diameter of the widened section. The thread is preferably formed there.

The tubular section of the connection part and/or the clamping component advantageously respectively comprise a multitude of openings distributed over the periphery, in order to simplify the growth of the blood vessel connection with the blood vessel. The openings extend as openings in the radial direction through the wall of the connection part and/or clamping component. Particularly preferably, they are designed as axially extending elongate holes. The openings may also serve for preventing a dying-off of the blood vessel tissue in the region in which the blood vessel is clamped between the connection part and the clamping component, by ensuring the supply of blood through the openings.

The individual components of the blood vessel connection are preferably manufactured of a biocompatible plastic. Such plastics have the advantage that the individual components may be manufactured in a comparatively simple and inexpensive manner with casting/molding methods used in plastics processing technology. Preferably, the blood vessel connection according to embodiments of the invention is formed from polyethylene terephthalate. However, a partial or even a complete design of metal is conceivable.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective exploded representation of a blood vessel connection for a vessel prosthesis in accordance with an embodiment of the invention; and

FIG. 2 is a longitudinal section view of the blood vessel connection according to FIG. 1 in the assembled condition.

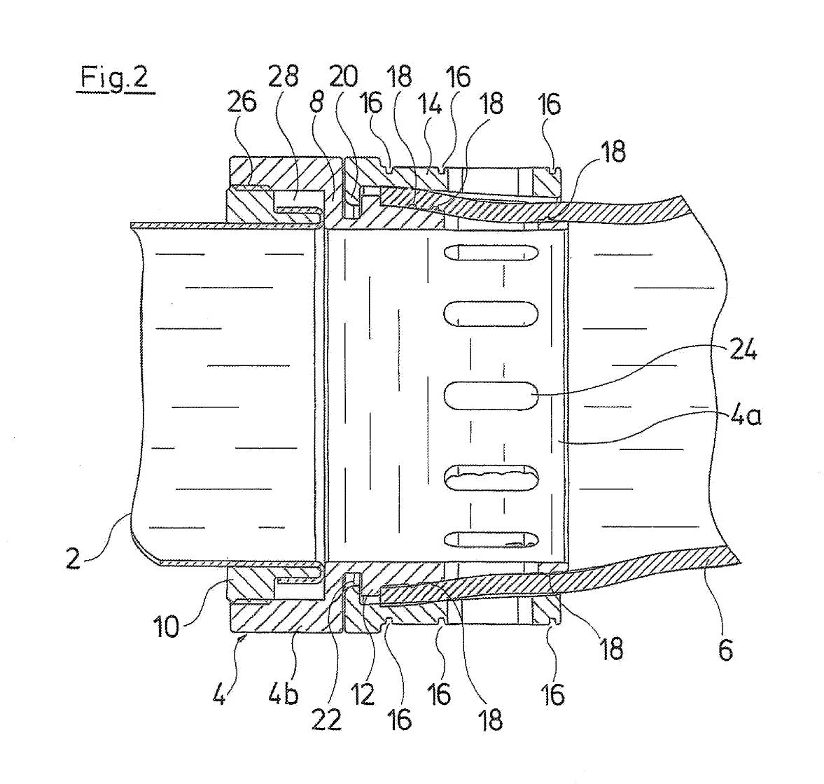

### DETAILED DESCRIPTION OF THE INVENTION

The blood vessel connection according to an embodiment of the invention, for a vessel prosthesis 2, comprises a connection part 4. The vessel prosthesis 2 may be connected on this connection part 4. Moreover, the connection part 4 may be introduced into an open end of a blood vessel 6. The connection part 4 on the inside is continuously hollow and in this manner forms an axial flow channel from a blood vessel 6 connected to the connection part 4 to the vessel prosthesis 2, which is likewise connected on the connection part 4.

The connection part 4 comprises two sections 4a and 4b, which differ with regard to their outer cross section. Thus, a shoulder 8 is formed on the connection part 4, at which shoulder the outer diameter of the section 4b is larger compared to the outer diameter of the section 4a. Corresponding to this, the cavity forming the flow channel through the connection part 4, i.e., the inner diameter, is also larger at the shoulder 8. The widened cavity in the region of the section 4b of the connection part 4 serves for receiving a fastening sleeve 10, with which the vessel prosthesis 2 is fixed on the connection part 4.

The section 4a of the connection part 4 serves for being introduced into the blood vessel 6, and thus for creating a flow connection from the blood vessel 6 to the blood vessel connection and the vessel prosthesis 2 connected thereto. Accordingly, the outer cross section in the region of the section 4a corresponds essentially to the inner cross section of the blood vessel 6 to be connected, wherein the outer cross section of the section 4a, proceeding from an annular projection 12, which is formed on the outer side of the section 4a and which serves as an abutment for the open end of the blood vessel 6, tapers in the direction of the free end of the section 4a. Thereby, the wall thickness of the connection part 4 is also reduced in the region of the section 4a, so that the end of the connection part 4 which is introduced into the blood vessel 6 represents an obstacle which may be neglected with regard to a possible thrombosis formation. Moreover, the cross-sectional tapering in the region of the section 4a simplifies the introduction of the connection part 4 into the blood vessel 6. The flow channel formed by the connection part 4 in the section 4a has a constant inner cross section which corresponds essentially to the inner cross section of the blood vessel 6 to be connected on the connection part 4, so that an almost shoulderless transition from the blood vessel 6 to the flow channel formed by the connection part 4 in the section 4a results.

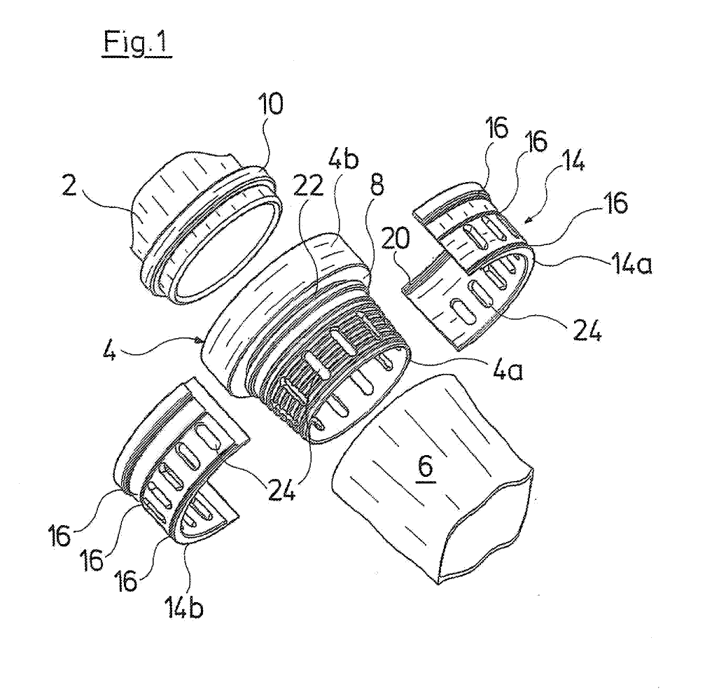

A sleeve-like clamping component 14 is provided on the connection part 4 for the force-fit fixation of the blood vessel 6 bearing on the outer side of the section 4a. The clamping component 14 is designed of two parts (see FIG. 1) and comprises two shell-like parts 14a and 14b which are semicircular in cross section, may be joined together in a simple manner and may be fixedly connected to one another, for example by a locking connection (not shown here). The length of the two parts 14a and 14b of the clamping component 14 is larger than the length of the region in which the blood vessel 6 overlaps the section 4a of the connection part 4.

Several grooves 16 running in the peripheral direction are formed on the outer side of the parts 14a and 14b of the clamping component 14 and are arranged such that, in the assembled condition of the clamping component 14, respective grooves 16 running annularly about the periphery of the clamping component 14 result for securing the connection of the parts 14a and 14b of the clamping component 14. Fastening threads, which are not represented and which after their free ends are tied together, likewise hold the clamping component 14 together, may be guided in these grooves 16.

Several annular projections 18 are formed on the outer side of the section 4a and press into the blood vessel 6 with the displacement of blood vessel tissue, in order to prevent a slippage of the blood vessel 6 on the section 4a in the axial direction of the connection part 4. The projections 18 are designed in a rounded manner and are comparatively short, in order to prevent an injury of the blood vessel tissue.

A slippage of the clamping component 14 in the axial direction of the connection part 4 is prevented by a projection 20, which is formed respectively at an axial end of the clamping components 14a and 14b on their inner side and which extends over the complete inner periphery. This projection is directed radially inward and in the assembled condition of the blood vessel connection engages into an annular recess 22, which is formed on the connection part 4 between the shoulder 8 and the projection 12.

A multitude of openings 24, in the form of long holes extending radially through the respective wall, are formed on the section 4a of the connection part 4 as well as on the clamping component 14, in a manner distributed over the respective periphery. These openings serve for simplifying a growth of the blood vessel connection with the blood vessel 6 and for preventing a dying of the tissue of the section of the blood vessel 6 which is clamped between the connection part 4 and the clamping component 14.

The fastening sleeve 10 may be screwed into the inside of the section 4b. For forming a thread pairing 26 with an inner thread formed on the inner side of the section 4b of the connection part 4, the fastening sleeve 10 comprises an outer thread which, in the screwed-in condition of the fastening sleeve 10, is arranged on the end of the fastening sleeve 10 which faces away from the shoulder 8 of the connection part 4. In the axial direction, following the outer thread of the fastening sleeve 10, the outer cross section of the fastening sleeve 10 tapers in a shoulder-like manner, so that an intermediate space 28 arises there between the inner wall of the widened section of the connection part 4 and the wall of the fastening sleeve 10.

The vessel prosthesis 2 is first led through the inside of the fastening sleeve 10, for fastening the vessel prosthesis 2 onto the connection part 4. At the end of the fastening sleeve 10 which faces the shoulder 8 in the screwed-in condition, a section of the vessel prosthesis 2 which projects beyond this end is pushed over the end, and subsequently the fastening sleeve 10 is screwed so far into the inside of the section 4b of the connection part 4, until the vessel prosthesis 2 is clamped between the shoulder 8 and the end-side of the fastening sleeve 10. The protruding section of the vessel prosthesis 2 is hereby received by the intermediate space 28 between the wall of the connection part 4 and the wall of the fastening sleeve 10. The constant inner cross section of the vessel prosthesis 2 corresponds to the inner cross section of the connection part 4 in the section 4a, so that the connection part 4 and the vessel prosthesis 2 fastened therein form a smooth flow channel with a constant inner cross section from the blood vessel 6 to a blood vessel which is connected to the end of the vessel prosthesis 2 facing away from the connection part 4, which is not represented in the drawing, or after the resection of a diseased section of the blood vessel 6, form a flow channel from a healthy section of the blood vessel 6 to a further healthy section of the blood vessel 6.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-14. (canceled)

15. A blood vessel connection for connecting a vessel prosthesis (2) with a blood vessel (6), the connection comprising a connection part (4) having a tubular section (4a) to be introduced into an open end of the blood vessel (6) to form a flow channel from the blood vessel (6) to the vessel prosthesis (2) connected on the connection part (4), a sleeve-shaped clamping component (14) for arrangement on an outer side of the tubular section (4a) to fasten the blood vessel (6) on the connection part (4), and a fastening sleeve (10) for fixing the vessel prosthesis (2) on the connection part (4), the connection part (4) having a cavity which forms the flow channel through the connection part (4) and a widened section (4b) which is shoulder-shaped and connects to the tubular section (4a), wherein the fastening sleeve (10) is screwable into the widened section (4b), wherein the widened section (4b) is dimensioned such that an inner wall of the vessel prosthesis (2) is flush with an inner wall of the flow channel formed by the connection part (4), and wherein the tubular section (4a) and the clamping component (14) have a plurality of openings (24) distributed about their respectively peripheries.

16. The blood vessel connection according to claim 15, wherein the clamping component (14) comprises two parts.

17. The blood vessel connection according to claim 16, wherein the two parts (14a, 14b) of the clamping component (14) are designed to form a locking connection with one another.

18. The blood vessel connection according to claim 15, wherein the clamping component (14) has at least one groove (16) formed on and extending around an outer periphery thereof.

19. The blood vessel connection according to claim 16, wherein the two parts (14a, 14b) of the clamping component (14) are connected by a hinge.

20. The blood vessel connection according to claim 15, wherein the clamping component (14) and the connection part (4) are connectable by a form fit.

21. The blood vessel connection according to claim 20, wherein the clamping component (14) comprises at least one radially inwardly directed projection (20) for engagement into at least one recess (22) formed on an outer side of the connection part (4).

22. The blood vessel connection according to claim 15, wherein the connection part (4) has at least one radially outwardly directed projection extending around an entire periphery of the connection part (4) and formed on the outer side of the tubular section (4a) which is introduced into the blood vessel (6).

23. The blood vessel connection according to claim 15, wherein an outer cross section of the connection part (4) tapers in a region of the tubular section (4a) in a direction of its free end.

24. The blood vessel connection according to claim 15, wherein the fastening sleeve (10) is dimensioned in an axial direction such that a vessel prosthesis (2) leading through the fastening sleeve (10) and overlapping an end of the fastening sleeve (10) which engages into the connection part (4) in a screwed-in condition is clampable between the end of the fastening sleeve (10) and a shoulder (8) of the connection part (4).

25. The blood vessel connection according to claim 15, wherein the connection is formed of polyethylene terephthalate.
